(19) United States
(12) Patent Application Publication
(10) Pub. No.: US 2007/0104645 A1
(43) Pub. Date: May 10, 2007

(54) Chelate based scaffolds in tumor targeting

(76) Inventors: Joseph R. Garlich, Westfield, IN (US); Robert G. Suhr, Greenfield, IN (US); Mary Patterson, Carmel, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 10/573,938

(22) PCT Filed: Sep. 30, 2004
(86) PCT No.: PCT/US04/32289

§ 371 Date: Jul. 25, 2006

Related U.S. Application Data
(60) Provisional application No. 60/507,427, filed on Sep. 30, 2003.

(51) Int. Cl. A61K 51/00; A61K 31/555; C07F 5/00

(52) U.S. Cl. 424/1.11; 514/184; 534/11; 534/15

(57) ABSTRACT

This invention relates to novel complexes that can be used to target tumor cells. The complexes include a ligand that can bind metal ions including radioactive lanthanide ions. The complexes can mimic α_{ν}β3 integrin receptor antagonists and deliver the complexed radioactive metals to the tumor cells.

### CROSS-REFERENCE TO RELATED APPLICATION

The present application claims the benefit of U.S. Provisional Application Ser. No. 60/507,427 filed on Sep. 30, 2003, which is hereby incorporated by reference in its entirety.

### GOVERNMENT RIGHTS

The U.S. Government has paid-up license in this invention and the right in limited circumstances to require the Patent Owner to license others on reasonable terms as provided by the terms of Contract No. 1R41CA92835-01 awarded by the National Cancer Institute.

### BACKGROUND OF THE INVENTION

In general this invention relates to novel complexes and their use to target tumor cells. More specifically, the present invention relates to novel complexes that chelate metal ions and deliver the metal ion to receptors on tumor cells and the endothelial cells found in neovasculature supporting tumor growth.

Cancer research has been increasingly focused on tumor vasculature as a potential target for new therapies. Agents such as angiostatin and endostatin have been discovered which can potentially prevent the formation of new blood vessels (angiogenesis) and thus prevent further growth of solid tumors.^{1,2}

More recently another approach has been described which seeks to take advantage of the differences between normal tissue vasculature and the new vasculature (neovasculature) supporting tumors for the purposes of selectively targeting of drugs to tumors. These differences in vasculature have been noted in the physiology³ of tumors as well as more recently at the molecular genetic level⁴ of endothelium tissue. Monoclonal antibodies (Mabs) that recognize tumor vasculature specific antigens have been labeled with the alpha-emitter isotope ²¹³Bi and found to extend the life-span of tumor laden mice.⁵ However, monoclonal antibodies as delivery agents in humans have significant hurdles in becoming therapeutic delivery agents.⁶ In particular, Mabs, proteins, and large polypeptides suffer from many problems as in vivo agents and, in fact, some research groups have given up work on angiostatin in favor of developing small molecules that would mimic the effects of the large proteins.⁷

Tremendous advances have been made in finding small molecules such as peptides that will target specific receptors in vivo. For example, Erkii Rusolahti and Renata Pasqualini of the Cancer Research Center at the Burnham Institute, La Jolla, Calif., have used phage display peptide libraries to find low molecular weight peptides containing the RGD (Arg-Gly-Asp) sequence that attach selectively to endothelial cells in the vasculature of tumors 40-80 times higher than to endothelial cells in other tissues.⁸ The tumor associated receptors for these peptides appear to be the α_{ν}β₃ integrins which are receptors for vascular growth factors.⁹ The α_{ν}β₃ receptor has been reported to be highly expressed on many tumor cells including osteosarcomas, neuoroblastomas, glioblastomas, melanomas, and carcinomas—lung, breast, prostate, and bladder.²⁵ The number of receptors per cell, an important consideration in targeting therapies where quantities of drug delivered are important, has been estimated to be up to 125,000 per expressing endothelial cell.²⁵ However, it should be noted that while α_{ν}β₃ integrin is selectively expressed in angiogenic blood vessels versus normal endothelial cells, there are other sites in vivo that also express this receptor under normal conditions (notably osteoclasts).²⁶ The RGD-containing peptide sequences isolated by Rusolahti, possessing high binding selectivity for the α_{ν}β₃ integrin receptor, have been tagged with anticancer drugs such as doxorubicin^{8,10} and shown to enhance the efficacy of the drug against human breast cancer xenografts in nude mice versus the unmodified doxorubicin control. This appears to be the first example of using the selective localization of a low molecular weight ligand binding to tumor vasculature-associated α_{ν}β₃ integrin to deliver a therapeutic anticancer drug.

The use of the peptide approach to bind with α_{ν}β₃ integrin receptors exploiting radionuclides as the toxiphore, targeting the neovasculature of tumors, has been proposed¹¹ but only limited work has been published.^{19,20} One study examined several radioiodinated cyclic RGD peptides, which were modeled after the previously optimized cyclo-(-Arg-Gly-Asp-D-Phe-Val-) pentapeptide system cyclo(-Arg-Gly-Asp-D-Phe-Val-) [abbreviated as cRGDfV, see FIG. 1, compound [symbol] ). For this cyclo-pentapeptide series, it was found that a hydrophobic amino acid in position 4 (D-Phe substitution) increases the receptor affinity whereas the position 5 (valine substitution) had little influence on the affinity. This series of cyclo-pentapeptides (including the iodinated tyrosine replacement for D-Phe analog called P2) were shown to be nanomolar inhibitors of the vitronectin receptor α_{ν}β₃ integrin. Moreover, they were selective for the α_{ν}β₃ integrin receptor over the α_{IIb}β₃ receptor which is a glycoprotein involved in platelet aggregation. However there was a loss of activity from the tumor site. These results indicate that from a therapeutic standpoint, there remains a reevaluation on this cyclo-pentapeptide system as a therapeutic agent.

Habner and coworkers have extended the use of this cyclic pentapeptide, as described in recent presentations, by attaching the radioisotopes F-18, ¹⁸⁸Re, ⁹⁰Y, and ^{99m}Tc to closely related derivatives of c(RGDfV) wherein the V (valine) has been replace by K (lysine) covalently modified on the epsilon-amino group^{23,24} to contain a moiety capable of binding the radioisotope. The published data^{23,24} showed a similar pattern of diminished absolute amount of isotope located at the tumor over time after initial uptake but accompanied by increasing tumor-to-blood ratios.

One drawback or disadvantage to using radioiodinated peptides such as the vascular targeting agents is their susceptibility to natural levels of peptidases and proteases which leads to extremely fast clearance rates from the bloodstream. While this may sometimes be useful for imaging purposes to yield a better target-to-nontarget ratio, it is unacceptable in a therapeutic approach as it lowers the absolute amount of drug reaching the target.¹² Additional problems exist with radioiodinated peptides as opposed to chelated-metal-labeled peptides and that is the radioiodinated peptides are converted to iodotyrosines and iodide both of which clear quickly from the targeted site making the agent unacceptable in a therapeutic setting.¹²

Investigators have studied the use of peptidomimetics to overcome the peptide limitations described above (fast clearance, metabolization) with notable successes. For example, β-peptides have been used with success to mimic peptides as demonstrated by a cyclic β-tetrapeptide as a mimetic of somatostatin.¹⁴ Another example is the use of nonpeptide-like templates used to present mimetics of individual key binding residues of peptides in their interactions with a receptor. The cyclic peptide bioactive somatostatin is represented in binding by a very different-looking mimetic based on β-D-glucose.^{15,16} Binding assay results support the hypothesis that the glucose template (scaffold)-based presentation of binding groups can mimic somatostatin's biological activity.

This same approach did not work as well in the area of designing peptidomimetics for the α_{ν}β₃ antagonist cyclo(-Arg-Gly-Asp-D-Phe-Val-) [abbreviated as cRGDfV, see FIG. 1. compound 1] based on a carbohydrate template. In this work of Nicolaou et al., they first determined the solution structure of cRGDfV by NMR¹⁷. Based on molecular modeling, Nicolaou proposed and synthesized a handful of cRGDfV analogs based on the pyranose carbohydrate ring system as a template. Unfortunately, little to no binding of these mimics to α_{ν}β₃ integrin was observed. It was suggested that there may exist subtle requirements for the active cyclic peptide conformation, which may not be fulfilled by these mimics as well as perhaps a lack of sufficient rigidity associated with the carbohydrate framework.¹⁷

Others have investigated peptidomimetics of cRGDfV (1) based on other templates. Benzodiazapines such as structure 2 (FIG. 1) have been found to be low-nanomolar inhibitors of vitronectin binding to α_{ν}β₃ integrin with a 10000-fold selectivity over undesirable inhibition of α_{IIb}β₃ receptor.²¹ In this case, the 1,4-benzodiazepine acts as a Gly-Asp mimic with the benzimidazole unit acting as an arginine mimic. Another RGD peptidomimetic selective inhibitor of α_{ν}β₃ integrin was identified³ (3, SC-68448, see FIG. 1) which showed up to 80% reduction in tumor growth in a mouse-based Leydig cell tumor model²². This molecule is simply an open chain analog presenting a guanidine moiety (arginine mimic) and a carboxylic acid (aspartic acid mimic) separated by a spacer group which allows for their presentation in a spatial arrangement that recognizes the α_{ν}β₃ integrin.

Given the drawbacks and approaches described above, it would be desirable to treat cancers that are highly expressing α_{ν}β₃ integrin by a small nonpeptide molecule that 1) possesses a built-in chelating agent complexed with a therapeutic radioactive metal ion in a stable fashion and 2) the resulting nonpeptide metal-ligand molecule possesses a high affinity and selectivity to the α_{ν}β₃ integrin because it was optimized with the metal complex as an integral and necessary part of the three dimensional arrangement of groups responsible for biological activity. The present invention addresses these issues and provides additional benefits and advantages.

### SUMMARY OF THE INVENTION

The present invention relates to novel composition and complexes and their preparation and use thereof to target tumor cells. While the actual nature of the invention covered herein can only be determined with reference to the claims appended hereto, certain forms and features, which are characteristic of the preferred embodiments disclosed herein, are described briefly as follows.

In one form, the present invention provides a composition that comprises a metal-chelating ligand. The metal-chelating ligand can be used to complex to a variety of metal ions. The metal-chelating ligand includes a tetraazacyclododecane macrocycle ring core. At least two non-identical substituents are covalently bonded to and extend from the ring core. Each of the at least two non-identical substituents contain a group capable of binding to a cell receptor. The substituents can be located at various positioned about the ring core and the substituents can be bonded to either the nitrogen or carbon atoms of the ring.

In another form the present invention provides a macrocylic complex chelated to a medicinally or therapeutic beneficial metal ion optionally with one two or more unique ligands terminating in or otherwise including a cell receptor binding group. The macrocylic complex can be used to deliver the metal ion to receptors on tumor cells and the endothelial cells found in neovasculature supporting tumor growth.

In another form, the present invention provides a composition that comprises a metal-chelating ligand including tetraazacyclododecane macrocycle having one or more alkyl carboxylic acids or salts thereof appended to the ring nitrogen(s) and a guanidine substituent covalently bonded to a ring nitrogen of the metal-chelating ligand via an alkyl linking group, an alkyl carbonyl linking group, or an alkyl amide linking group. The alkyl groups of the alkyl linking group, the alkyl carbonyl linking group, and the alkyl amide linking group can be a straight chain, a branched chain, cyclic or aromatic hydrocarbyl group having between 1-6 carbon atoms, and can be substituted with one or more of the following substituents: hydrogen, a C1-C4 alkyl, branched alkyl, or aromatic or heteroaromatic group. The heteroaromatic atom or moieties that can be attached to the alkyl or the aromatic group include nitrogen, oxygen, sulfur, halogens, amines, amides, guanidine, carboxy, carbonyl, hydroxyl, sulfoxy, sulfoxide and mixtures of these groups. The macrocycle can have two or more potential chelating arms extending from the basic ring structure. The potential chelating arms can be at differing positions relative to each other about the ring (for example in the 1,4 nitrogen substitution pattern or 1,7 nitrogen substitution pattern). Further, the chelating arms can terminate in different binding groups or atoms. Non-limiting examples of binding groups/atoms include amines, amides, carbonyl, oxo, carboxy, and guanidine.

In another form, the present invention provides a composition that comprises a metal-chelating ligand including tetraazacyclododecane macrocycle having two or more alkyl carboxylic acids or salts thereof appended to the ring nitrogen(s), and two or more non-identical α_{ν}β₃ receptor binding ligands covalently bonded to a ring nitrogen or carbon of the metal-chelating ligand via an alkyl group linking group, an alkyl carbonyl linking group, or an alkyl amide linking group.

In yet another form, the present invention provides a method of inhibiting tumor cell growth. The method comprises administering to the tumor cells an effective amount of a composition including a compound having a metal-chelating ligand including tetraazacyclododecane macrocycle having two or more alkyl carboxylic acids or salts thereof appended to the ring nitrogen(s), and two or more non-identical α_{ν}β₃ receptor binding ligands covalently bonded to a ring nitrogen or carbon of the metal-chelating ligand via an alkyl group linking group, an alkyl carbonyl linking group, or an alkyl amide linking group.

In still yet another form, the present invention provides a method of inhibiting tumor cell growth. In this method, a metal-chelating ligand including tetraazacyclododecane macrocycle with two or more alkyl carboxylic acids or salts thereof is appended to the ring nitrogen(s), and a guanidine substituent covalently is bonded to a ring nitrogen of the metal-chelating ligand via an alkyl linking group, an alkyl carbonyl linking group, or an alkyl amide linking group.

### BRIEF DESCRIPTION OF THE DRAWINGS

FIG. 1 illustrates the structure of c(RGDfV) and two non-peptide mimetics.

FIG. 2 illustrates DOTA and two c(RGDfV) mimics, based on DOTA modifications.

FIG. 3 illustrates four base structures for use as α_{ν}β₃ integrin antagonists in a multivalent construct and examples of linker/spacer modules.

FIG. 4 illustrates a scheme for the solid-phase synthesis of one of the DOTA based c(RGDfV) mimetics, DOTA-RXG (R²═R³═H; R⁴═CH₂COOH; R⁵═CH₂CH₂-p(Ph)—NH(C═NH)NH₂) as a single member of a combinatorial library.

FIG. 5 is a scheme illustrating a strategy for the preparation of chiral aminoesters for use in combinatorial synthesis in FIG. 3 in accordance with the present invention.

FIG. 6 is a scheme illustrating a strategy to achieve stereochemical control at each chiral acetate arm position such as DOTA-G in accordance with the present invention.

FIG. 7 illustrates the conceptual design of chelabodies based on DOTA-type chelating agents presenting a tetravalent binding arrangement aimed at α_{ν}β₃ integrin antagonism in accordance with the present invention.

FIG. 8 illustrates representative examples of linker/spacer modules for α_{ν}β₃ integrin antagonist each with different linking groups.

FIG. 9 is a scheme illustrating one route for the solid phase syntheses of the macrocyclic chelator RGD mimetics in accordance with the present invention.

FIG. 10 is a synthetic scheme for the amine containing chain extenders to attach to the base DOTA scaffold in accordance with the present invention.

FIG. 11 illustrates a synthetic route for the 1,7-substituted tetraazamacrocycle (14).

FIG. 12 illustrates the general syntheses of a 1,4 disubstituted DOTA-type agent.

FIG. 13 illustrates the general synthetic scheme for the preparation of an amide 1,7 disubstituted DOTA-type agent.

FIG. 14 illustrates the general preparation of alpha bromo diacids used to attach the variable length linker groups to the DOTA scaffold in accordance with the present invention.

FIG. 15 illustrates the general preparation of alpha bromo amides with BOC protected amines used to attach variable length linker groups to the DOTA scaffold in accordance with the present invention.

FIG. 16 is a reaction scheme for the typical synthesis of a DOTA based RGDS mimetic that showed promise as a α_{ν}β₃ integrin receptor antagonists.

FIG. 17 is a table listing various examples of DOTA based macrocyclics that can be prepared and complexed with a metal ion in accordance with the present invention.

### DETAILED DESCRIPTION OF THE INVENTION

For the purposes of promoting an understanding of the principles of the invention, reference will now be made to the embodiments illustrated herein, and specific language will be used to describe the same. It will nevertheless be understood that no limitation of the scope of the invention is thereby intended. Any alterations and further modifications in the described complexes, synthesis, and uses of the complexes thereof, and any further applications of the principles of the invention as described herein, are contemplated as would normally occur to one skilled in the art to which the invention relates.

The present invention provides a composition or complex that comprises a metal-chelating ligand. The metal-chelating ligand includes a tetraazacyclododecane macrocycle ring core. At least two non-identical substituents covalently bonded to and extend from the ring core, wherein each of the at least two non-identical substituents contain a group capable of binding to a cell receptor.

The present invention also provides chelating agent moiety itself as a template upon which to place the α_{ν}β₃ integrin binding moieties (specifically an acidic group and a basic group existing as a negatively charged and positively charged species respectively at physiological pH) in a spatial arrangement that mimics the well known α_{ν}β₃ integrin antagonist c(RGDfV) 1. The synthesis involved in this approach is detailed below. Expanding on this approach is the use of the chelating agent as the platform from which to tether multiple copies of a selective α_{ν}β₃ integrin-binding moiety such as c(RGDfV). This novel, multivalent approach is explored combinatorially to find the optimum distances between the multiple copies of the binding moiety and to study the effect of different spacing groups on the binding of the resulting construct with integrins.

The synthesized molecules that mimic the binding of monoclonal antibodies are called chemobodies.³⁵ As used herein, the term “chelabodies” describes chelates (metal-ligand complexes) that mimic the binding of monoclonal antibodies. Thus, chelabodies represent a subset of chemobodies wherein the chelate is a design feature that causes arrangement of the binding motifs in the appropriate spatial arrangement to give antibody-like multivalent binding. Compounds described herein fit into this new category of chelabodies.

FIG. 2 illustrates the chelating agent DOTA, (1,4,7-10-tetraazacyclododecane-tetraacetic acid), which is known to form kinetically inert complexes with the lanthanides²⁸ and the resulting complexes are considered conformationally rigid.²⁹ The resulting complexes are overall negatively charged at physiological pH when complexed with a metal ion. Examples of metal ions that can be complexed to the DOTA based scaffold, as described herein, include medically useful metal ions metals ions used for x-ray contrast agents, MRI contrast agents, and radioactive complexes and include those metals cited in Chemical Reviews, “Medicinal Inorganic Chemistry”. vol. 99, No. 9,1999. Non-limiting examples of metal ions for use in the present invention include ions of: La, Ce, Pr, Nd, Pm, Sm, Eu, Gd, Th, Dy, Ho, Er, Tm, Yb, Lu, Y and Sc. The preferred X-ray contrast and MRI imaging metal ion is Gd⁺3. The preferred radioactive metal ions are the ions of: ¹⁵³Sm, ¹⁶⁶Ho-166, ⁹⁰Y-90, ¹⁴⁹ Pm, ¹⁵⁹Gd, ¹⁴⁰La, ¹⁷⁷LU, ¹⁷⁵Yb, ⁴⁷Sc, and ¹⁴²Pr. One of the attractive features of a complex utilizing lanthanides as the metal ion is attributable to the variety of radioactive lanthanides in use in nuclear medicine (¹⁵³Sm⁺³, ⁹⁰Y⁺³, ¹⁶⁶Ho⁺³) with differing half-lives and beta-particle energies.

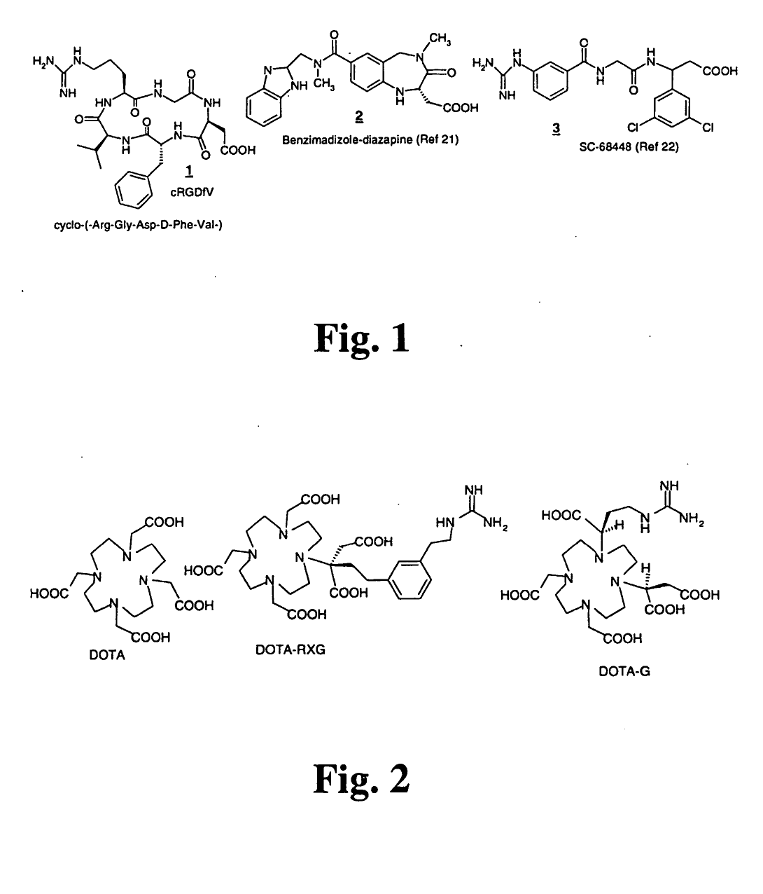

The molecular models of DOTA complexes indicates that DOTA is similar in size to the peptide ring α_{ν}β₃ integrin antagonist c(RGDfV) (compound 1, FIG. 1). Therefore suitable c(RGDfV) mimics are described in the present invention by judicious substitution patterns on the DOTA backbone. For example, molecular modeling indicates that structure (DOTA-RXG) (see FIG. 2) when complexed with Y⁺³ would place the guanidine and carboxylic acid in a similar spatial arrangement as that found for the guanidine of the arginine and the carboxylate of the aspartic acid residues in c(RGDfV).²⁹ Likewise, from modeling estimates DOTA-G (upon complexation with Y⁺³, see FIG. 2) appears to also satisfy the spatial requirements of the binding moieties of c(RGDfV).²⁹ Structure DOTA-RXG represents a single arm attachment and structure DOTA-G represents adjacent chelating arm modifications. It should be noted that modeling indicates that similar achievement of a c(RGDfV) mimic using modifications of acetate arms that are not adjacent would be difficult unless extremely large and conformationally floppy spacer groups are used. Nevertheless, the present invention contemplates such modifications.

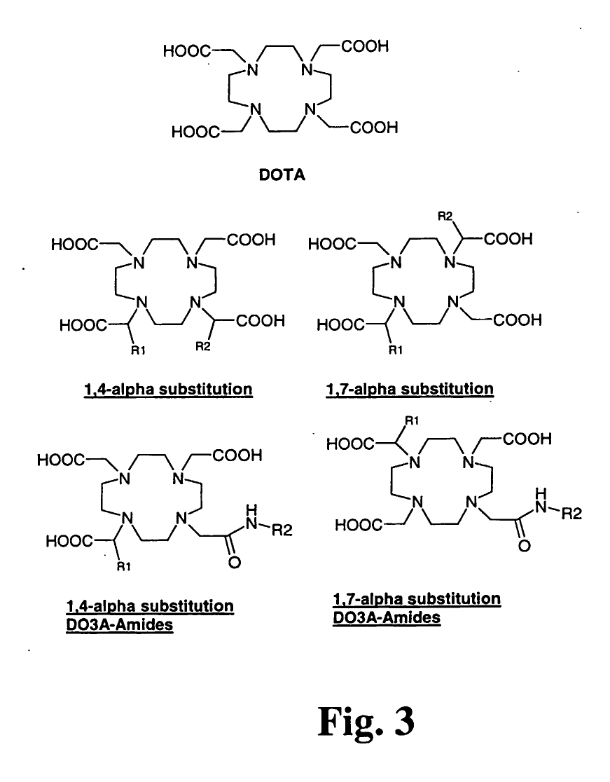

Modeling Studies: Molecular modeling studies support the use of DOTA complexes as chelate scaffolds.³⁰ The crystal structure of the extracellular segment of the α_{ν}β₃ receptor with an arginine-glycine-aspartic (RGD) amino acid containing ligand was published in the Apr. 5^{th}, 2002 issue of Science. The modeling studies used the crystal structure of the basic group of arginine (of the bound RGD ligand) relative to the acidic aspartic acid group to support the following: 1) a different metal ion size in the metal-DOTA complex does not significantly affect the spatial dispositions of the acetate arm substituents (allowing differently sized metals, i.e. from Y⁺³ to Ho⁺³ and therefore allow use of different therapeutic radioisotopes yet each still providing a same RGD mimetic); 2) the substitution pattern of 1,4 vs. 1,7 on the tetraazamacrocyclic ring using mono-substituted acetate arms both give plausible candidates for RGD mimetics (as depicted generically by structures labeled as the 1,4-alpha substitution and 1,7-alpha substitution respectively in FIG. 3); 3) the different stereoisomers due to the chirality introduced by a substituent on the chelating acetate arm points the substituent into different space, but depending on how one orients the complex into the α_{ν}β₃ receptor site, all possible stereoisomers can be positioned to mimic RGD; and 4) conversion of one of the chelating arms to an amide instead of carboxyl leads to additional novel compositions that show the substituents to be in a spatial arrangement to be potential RGD mimetics (this is depicted generically by structures 1,4-alpha substitution DO3A-Amides and 1,7-alpha substitution DO3A-Amides, respectively in FIG. 3). Thus, some preferred embodiments of the DOTA scaffolds predicted by modeling are those represented by structures 1,4-alpha substitution and 1,7-alpha substitution, 1,4-alpha substitution DO3A-Amides and 1,7-alpha substitution DO3A-Amides in FIG. 3 where the R1 and R2 comprise a basic amino group and an acidic group with varying length of attachment to the acetate arm.

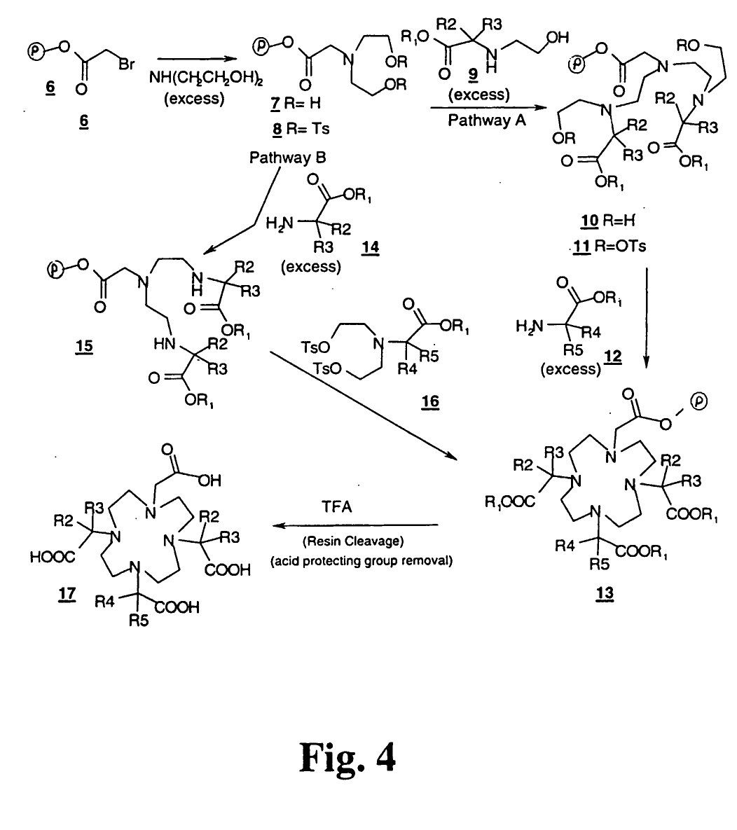

There are numerous other possible substitutions on the acetate arm besides those shown in DOTA-RXG and DOTA-G, which could restrict rotation even further to provide additional pre-organization to mimic c(RGDfV). Additionally, there are many additional groups that can serve as carboxylate mimics and guanidine mimics according the present invention. Therefore the present invention also provides a library of compounds similar to DOTA-RXG, guided by molecular modeling, via the solid-phase combinatorial chemistry route proposed in FIG. 4.

FIG. 4 illustrates a scheme for the solid-phase synthesis of one of the DOTA based c(RGDfV) mimetics, DOTA-RXG_(R²═R³═H; R⁴═CH₂COOH; R⁵═CH₂CH₂-p(Ph)—NH(C═NH)NH₂) as a single member of a combinatorial library. In FIG. 4 the circled P represents the solid phase resin, Wang resin in this case. However, the use of Rink amide resin is also used and would give a DOTA-based chelator wherein one of the chelating acetate arms is a —CH₂C(O)NH₂ group upon cleavage from the resin. These types of chelators are suitably stable for in vivo use.²⁹ An additional advantage of this monoamide from Rink amide resin would be that the resulting complex with trivalent lanthanides gives a neutral complex core molecule. This could have important in vivo biodistribution advantages.

The synthetic scheme (FIG. 4) to prepare these molecules illustrates two pathways to get to the same desired substituted DOTA chelator, 17. This synthetic scheme represents the first on-resin synthesis of the medically important tetraazacyclododecane ring system. Thus this work illustrates an exciting combinatorial chemistry methodology advance in the area of chelation based inorganic medicinal chemistry. By using R²═R³═H the synthesis as shown in FIG. 4 simplifies to only one chelator arm substituted with two moieties. The stereochemistry is not shown in FIG. 4 but the use of the proper enantiomer of 12, which can be isolated, allows the deliver the desired stereoisomer as shown in structure DOTA-RXG in FIG. 2.

One of the key building units to get to structures like DOTA-RXG via the route shown in FIG. 4 is a chiral unnatural amino acid derivative. A diverse collection of these disubstituted glycine derivatives can be prepared in solution phase or solid phase by the UPS (unnatural peptide synthesis) route pioneered by O'Donnell.^{31,32} This procedure is shown in FIG. 5 and lends itself to automation.³³ The different enantiomers resulting in synthesis are separated using chiral chromatography. There are methods to perform the chemistry shown in FIG. 4 wherein either R⁴ or R⁵ is hydrogen with significant stereoselectivity (80-90% ee). However, in a preferred embodiment of the present invention, a purity of greater than 95% ee can be achieved if chiral separation is performed at this stage. This will be performed using HPLC or SFC methodology.

With the inputs 12 (and 14, which can be the same or different from 12, derived from the same chemistry) in hand, then the library production protocol based on structure DOTA-RXG can be developed. Because of the way the synthesis is developed, it is possible to make an analog of DOTA-RXG where each of the three acetate arms contain one copy of the RGD mimic structure by making 12 and 14 the same amino ester. This trivalent species, by benefit of compact presentation of three copies of the RGD mimic structure, possesses interesting properties as discussed more fully below.

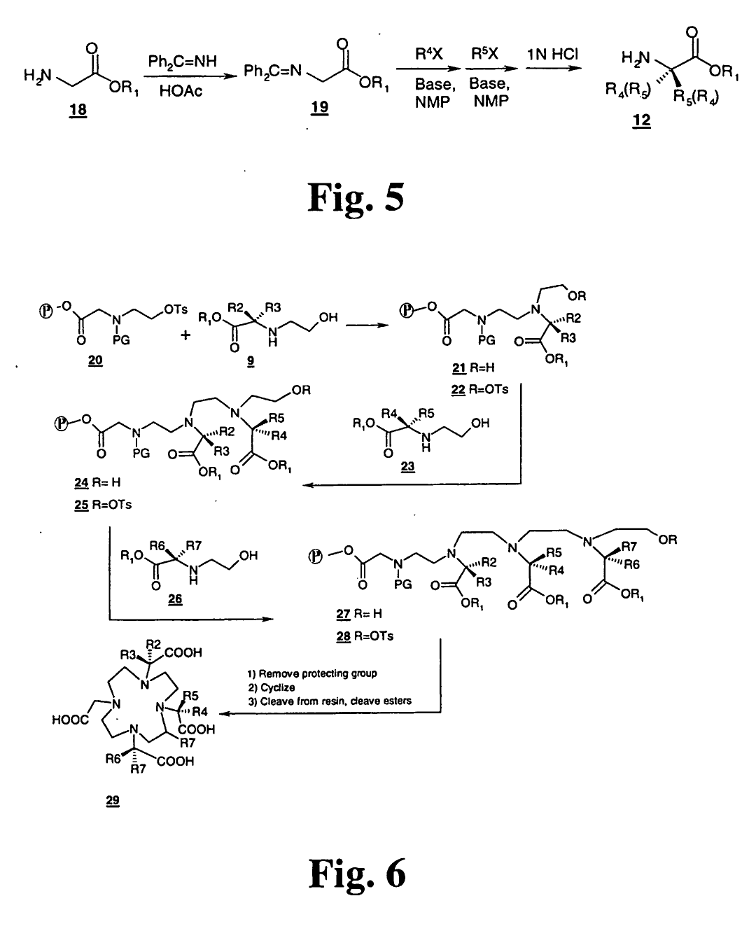

In order to access desired target molecules such as DOTA-G, a different synthesis route is utilized since two identical molecules of the amino ester are incorporated in either pathway A or pathway B in FIG. 4. This uncontrollable dual incorporation precludes introducing the needed stereochemistry at both sites, i.e. only one acetate substitution pattern will have the correct configuration. To address the desired access to molecules like DOTA-G and to give complete control over the stereochemistry of all 6 substituents on the chelating acetate arms, the synthetic protocol shown in FIG. 6 can be prepared and evaluated. The amino alcohols 9, 23, and 26 are prepared from the corresponding unnatural amino esters prepared by the method shown in FIG. 5 and purified to get the single isomer. One method of preparing these aminoalcohols could make use of resin bound ethylene glycol wherein the amine of the amino ester (such as 12) displaces the activated non-resin bound hydroxyl of the ethylene glycol. The PG (protecting group) on the nitrogen of FIG. 6 is selected to ensure orthogonal stability. The protecting group can be selected from a variety of groups such as FMOC, NOSYL, or trifluoroacetamide.

The chelator scaffolds (chelabodies) address the shortcomings described previously for a tumor neovasculature seeking agent. The positive attributes for this system are 1) nonpeptide in nature so not prone to metabolism; 2) incorporates a kinetically inert lanthanide complex which allows for a potential range of radioisotopes having varied particle energies and half-lives and yet produced commercially (Sm-153, Ho-166, and Lu-177); 3) rigid backbone (cyclododecane ring system locked into place upon chelation) upon which to place appropriately spaced recognition/binding groups; and 4) the complex containing the toxiphore (radioactive metal ion) is part of the core rigidifying structure so no additional conjugation chemistry is required, i.e. the compound from screening will not need to be further modified to label with a radioactive isotope.

Another aspect of the present invention targets a secondary binding site in the receptor; this is made possible by the chemistry routes disclosed herein that allow for differential substitutions to be made on the DOTA scaffold. In this case one acetate arm of DOTA can be covalently attached to a known α_{ν}β₃ antagonist molecule and a different acetate arm of that same complex would be attached to a group capable of binding to a nearby site in the α_{ν}β₃ binding pocket for example an electrophilic group reacting with the nucleophilic sulfhydryl group known to exist in the vicinity of the α_{ν}β₃ binding pocket.

Monoclonal antibodies are known for their exquisite selectivity and high binding affinity. These attributes arise in part because antibodies are divalent and in some cases multivalent in their binding with proteins or receptor surfaces. Nature has used multivalent binding to overcome weak binders in order to make strong attachments.³⁵ Multivalency, simultaneous attachment of two or more binding sites on one molecule (drug) to multiple receptor sites on another (cell surface), is a new approach to drug design according to George M. Whitesides of Harvard University.^{35,36} This multivalent approach has not yet been applied to ligands aimed at binding the integrins, although Burgess has disclosed a cyclic sequence c(RDGRGD) that could be considered a dimer of RDG.³⁷ Surprisingly this ligand possessed excellent selectivity and antagonistic activity towards α_{ν}β₃ integrin.

This area of multivalent drug design is where the term “chemobody” has been coined to describe synthesized molecules that mimic the binding of monoclonal antibodies.³⁵ The chelabodies of the present invention (chelates including metal-ligand complexes) mimic the binding of monoclonal antibodies. Thus, chelabodies represent a subset of chemobodies wherein the chelate is a critical design feature that causes arrangement of the binding motifs in the appropriate spatial arrangement to give antibody-like multivalent binding.

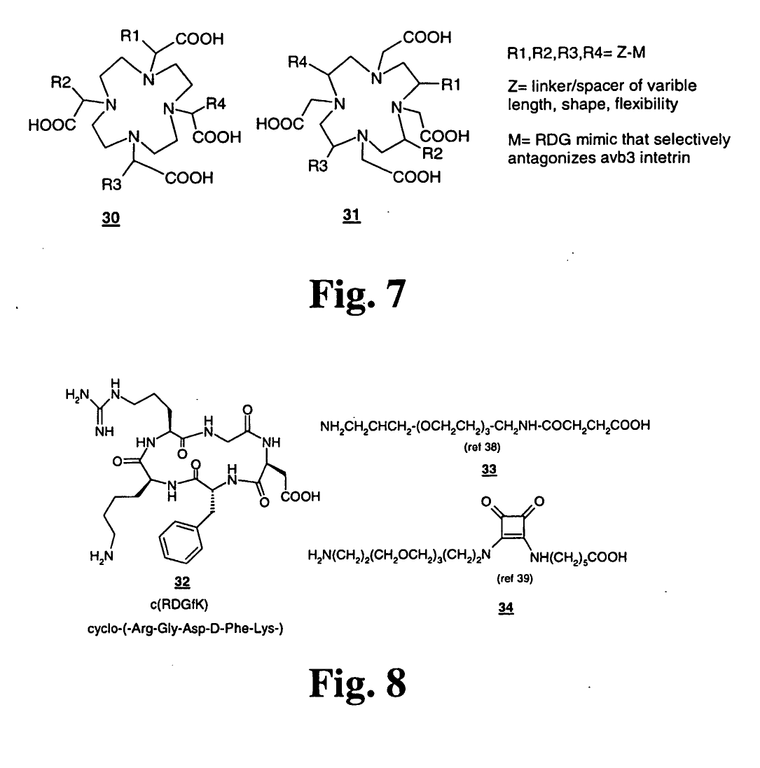

The present invention also contemplates the design, synthesis, and evaluation of multivalent presentations of α_{ν}β₃ integrin antagonists based on the DOTA template. This is illustrated conceptually in FIG. 7 where either four substitutions are made on the chelating arms (30) or situated around the macrocyclic ring (31). The present invention also contemplates the use of mixed species where some substitution is on the acetate arms and some is on the backbone carbons. One of the foci of these approaches is for the R groups to contain, preferably at their terminus, a moiety that is an α_{ν}β₃ integrin antagonist although the concept is applicable to any receptor wherein the binding requirements of the receptor antagonists are known for example from inspections of the crystal structure of the receptor alone or with antagonist bound in the receptor site.

A preferred terminal group includes a moiety that induces internalization of the bound ligand into the cell and methods are described to assess this property (see below Biological Evaluations). In order to prove the concept involved here, in one embodiment, the invention uses known antagonists at the terminal binding positions. For example, the known antagonist c(RGDfK) (32) has been described and is amenable to capping off the “R” arms to provide a suitable multivalent antagonist construct. The linker/spacer arms can be similar to those described in the literature for multivalent constructs, some of which are illustrated in FIG. 8. One embodiment that provides basic linker arms uses the reaction of carboxylic anhydrides with a nucleophile such as nitrogen on the arm stub and then couples a diamine with the resulting free carboxylic acid. This procedure is amenable to solid-phase synthesis to prepare linker arms that are all the same.^{38,39} Applying this strategy to the compounds requires only that some of the substituents (R2, R3, R4, R5, R6, R7) on the arm building blocks (9, 12, 14, 16, 23, 26) contain a masked electrophile (to react with amines for example) or nucleophile (to couple with carboxylic acids for example) that can be deprotected and then elaborated into a linker/spacer module for endcapping with antagonists such as 32. This approach takes advantage of the chemistry outlined in FIGS. 5 and 6 to give essentially trivalent constructs (i.e. one per each substituted chelator arm). One skilled in the art will appreciate the fact that a large number of different constructs can be prepared by varying the nature and length of the arms.

The present invention provides a large a combinatorial library of such constructs and assesses the library's members for the biological binding and performance (in vitro binding and whole cell assays) to determine improvements in tumor cell localization.

Biological Evaluations

Assay-In Vitro: The ELISA-type in vitro testing for competitive binding of test ligands with α_{ν}β₃ integrin is well established as are the methods to obtain the needed starting materials: vitronectin, α_{ν}β₃ integrin, fibrinogen, and α_{ν}β₃ integrin.^{19,22,27,41,42,43} Briefly, the solid-phase competitive displacement in vitro assay test comprises: 1) coating 96-well plates with α_{ν}β₃ integrin receptor (or α_{IIb}β₃ integrin receptor to determine selectivity), 2) washing sequence including 1% BSA, 3) exposure to various concentrations of test compound containing biotinylated vitronectin (or biotinylated fibronectin)^{t}9 for 2 hours, 4) washing sequence, and finally 5) detection of biotin present using reporter-labeled anti-biotin antibody. Initially this testing is performed on nonradioactive metal ion complexed with the newly synthesized compounds and is accomplished in a medium-throughput mode.

Assay-In Vitro Whole Cell Internalization Studies: A recently published method is described to determine internalization of integrins which are thought to occur via endocytosis.⁴⁴ The approach used in the present invention does not necessarily measure internalization (which requires anti-ligand antibodies) but exposes integrin expressing cells to the newly synthesized ligands and then determines the degree of binding by aggressive exposure to competitive ligand and various washes. Since all of newly synthesized molecules chelate radioactive metal ions, these radioactive metal complexes are easily determined as either cell associated or easily removed. Ultimately the location of the ligands is less important than ensuring that the antagonists stay bound to the cell surface so that in vivo they are able to deliver the desired radiation dose.

Animal Studies: The in vivo evaluation of the best in vitro active compounds on animals is performed following the procedures recently published in the area of nuclear medicine.¹⁹ These animal results using human tumors implanted into immune-compromised mice provide biolocalization data. The animals are sacrificed to quantitate the tumor and normal tissue uptake. The tumors and cell line for the tests are the melanoma line, WM164, which is available from ATTC.

For the purpose of promoting further understanding and appreciation of the present invention and its advantages, the following Examples are provided. It will be understood, however, that these Examples are illustrative and not limiting in any fashion.

### EXAMPLES

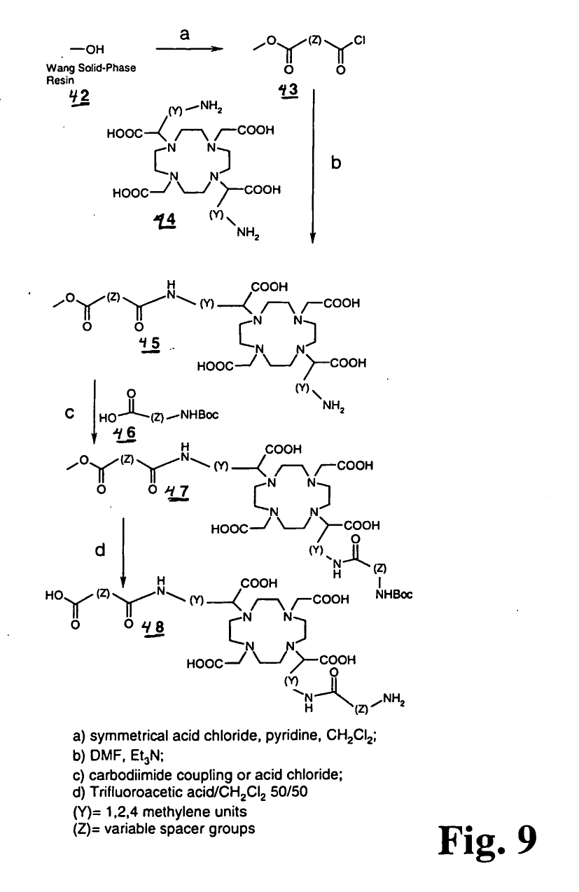

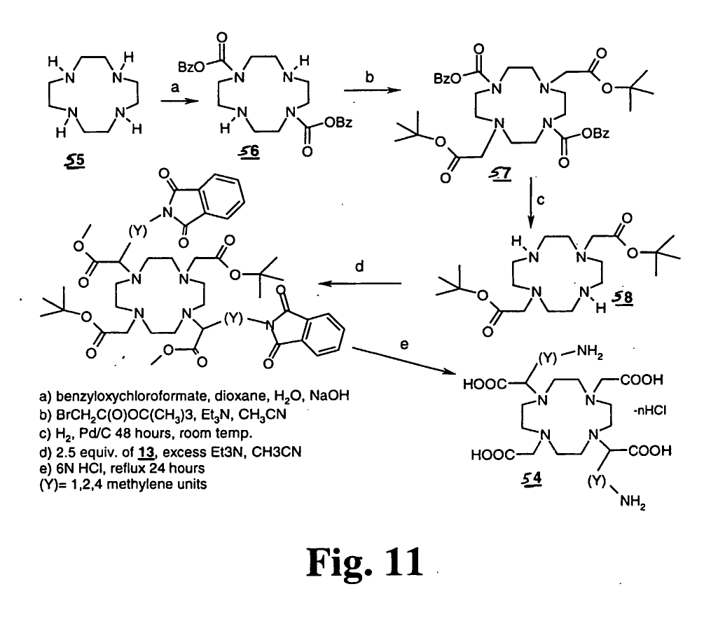

FIG. 9 illustrates one synthetic route that uses a solid phase approach for the synthesis of the DOTA-based multidentate metal ligand. (This route can be utilized to synthesize either 1,4 or 1,7-disubstituted analogs although only the 1,7 example is shown) This route starts with Wang solid phase resin 42 and utilized attachment of a symmetrical diacid 43 to the resin (representing the first variable input). The diacid 43 was then coupled with key bis-amine intermediate 44 via one of the amine groups to give 45. It should be noted that intermediate 44 represents a variable in chain length and orientation (i.e. 1,7 vs. 1,4 substitution on the macrocycle ring). Complete removal of all the protecting groups was effected in 6N HCl with heating for extended times to give the key bis-amine 44 as depicted in FIG. 11, which can then be attached to resin 42 to provide 45. The other amine group of 45 can be left as-is or reacted further with a protected amino acid 46 to extend out the chain length to give 47. This construct was then cleaved from the resin by exposure to trifluoroacetic acid which also deprotected the various amine and carboxy protecting groups to yield the macrocyclic chelator containing RGD mimetic groups 48. This synthetic route yielded sufficient quantities of the key intermediates for further elaboration.

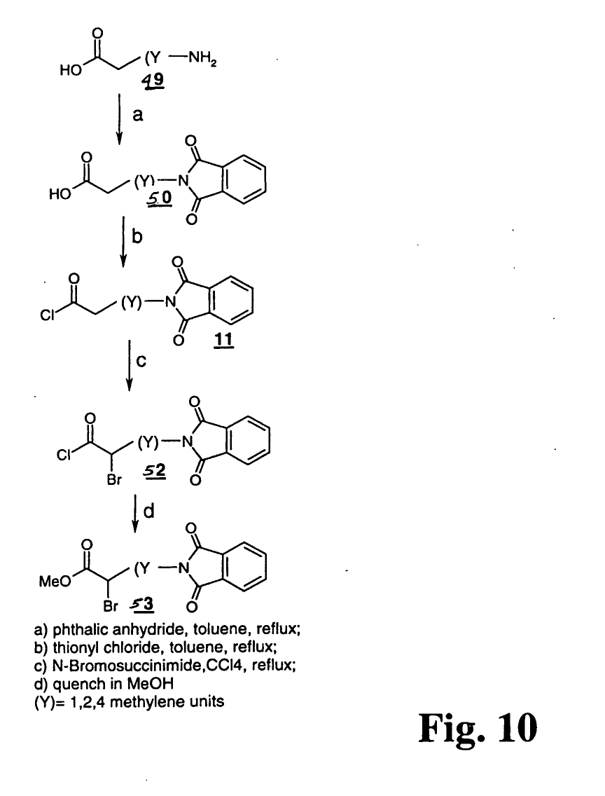

For the key pieces a series of 3-bromo-compounds 53 in multigram quantities were successfully prepared via the method shown in FIG. 10. Starting amino acids 49 were protected with a phthalimido group, then converted to the acid chloride, brominated, and then quenched in methanol. The resulting products were purified by preparative HPLC.

It should be noted that the yield of 53 where (y=3 methylene units) was low. However, the reaction of excess bromo-compounds 53 with the disubstituted cyclen 48 provided good yields of the tetra-substituted cyclen 54.

Then the 1,7-substituted tetraazamacrocycle precursor 54 was prepared in multigram quantities by the route shown in FIG. 11. The synthesis started with a commercially available cyclen 55.

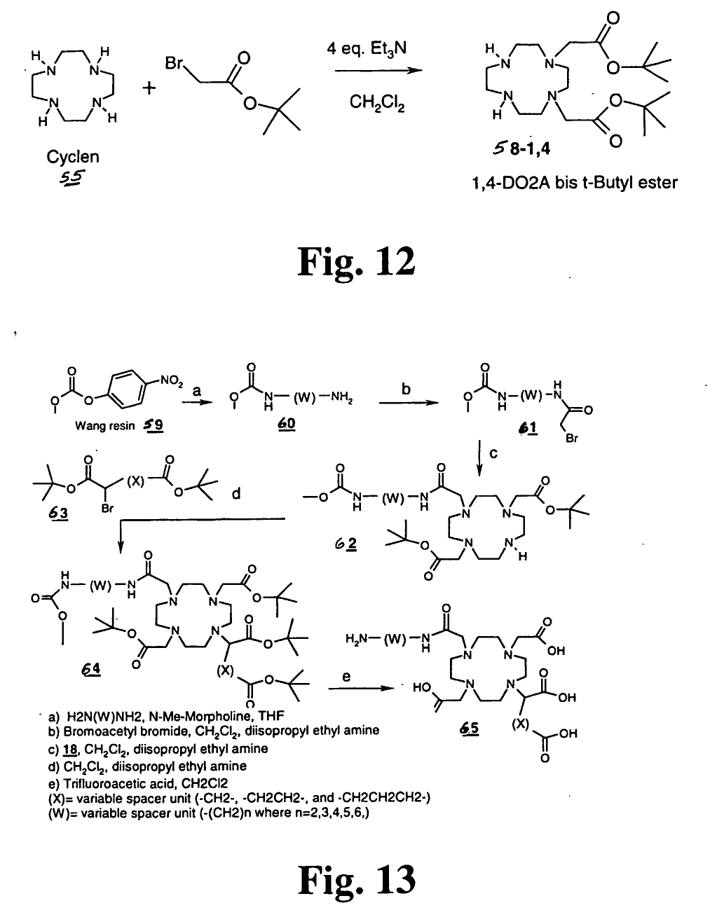

A novel synthesis of the 1,4-substituted version or isomer of 1,7-substituted compound, 1,4-DOTA bis t-butyl ester 58 (designated 58-1,4) was developed shown in FIG. 12. This procedure began by reacting cyclen 55 (commercially available) with t-butyl-bromoacetate (commercially available) in methylene chloride in the presence of triethylamine followed by extractions to isolate the products. Surprisingly large quantities of the 1,4-bis-substituted product was predominately formed. The reaction mixture was concentrated to very low volume, dissolved in methanol, diluted with equal volume of water, and placed in a hood to slowly evaporate. As the solution slowly air dried, the amines therein formed the carbonate salt (from carbon dioxide in the air) as a white solid. The different substitution products exhibited differential solubility in methanol depending on their substitution amount. The 1,4 disubstituted DO2A bis ester carbonate salt was observed to be insoluble in water but the DO1A ester carbonate salt was determined to be soluble in water. This allowed separation via water solubility and then neutralization of the carbonate salt (acidification first using HCl and then basification using excess aqueous sodium hydroxide and extraction into methylene chloride) to yield the reactive free base of DO2A with the 1,4-substitution pattern (58-1,4).

Because the modeling studies support the rationale of preparing compounds such as 65 as RGD mimetics, another synthesis using solid phase resin was developed as shown in FIG. 13. Although the synthesis was successfully completed for RGD mimetic 65, the solid phase results were highly variable when the different inputs were varied. Therefore this route was deemed less attractive for library production.

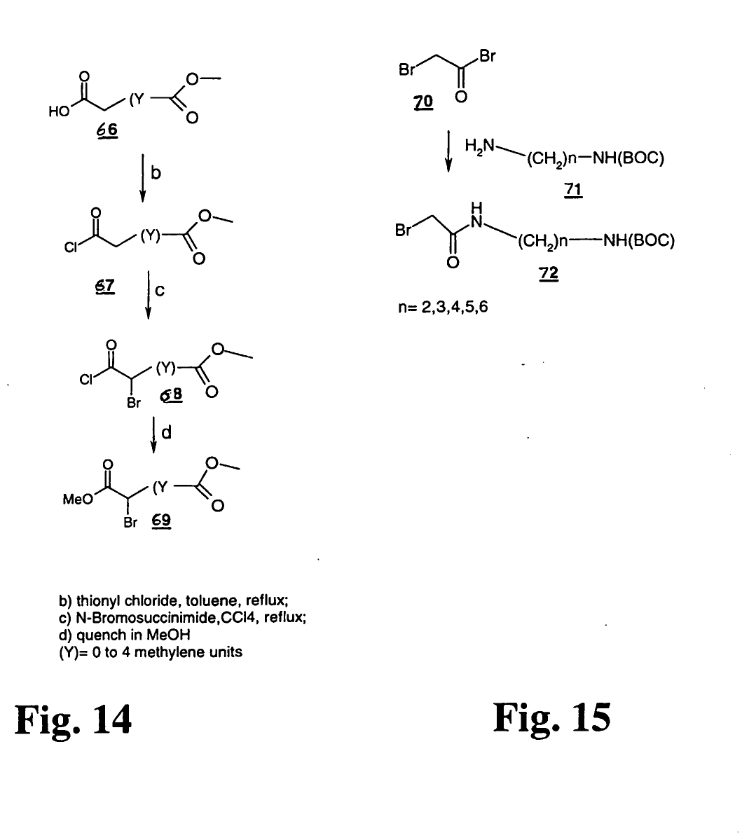

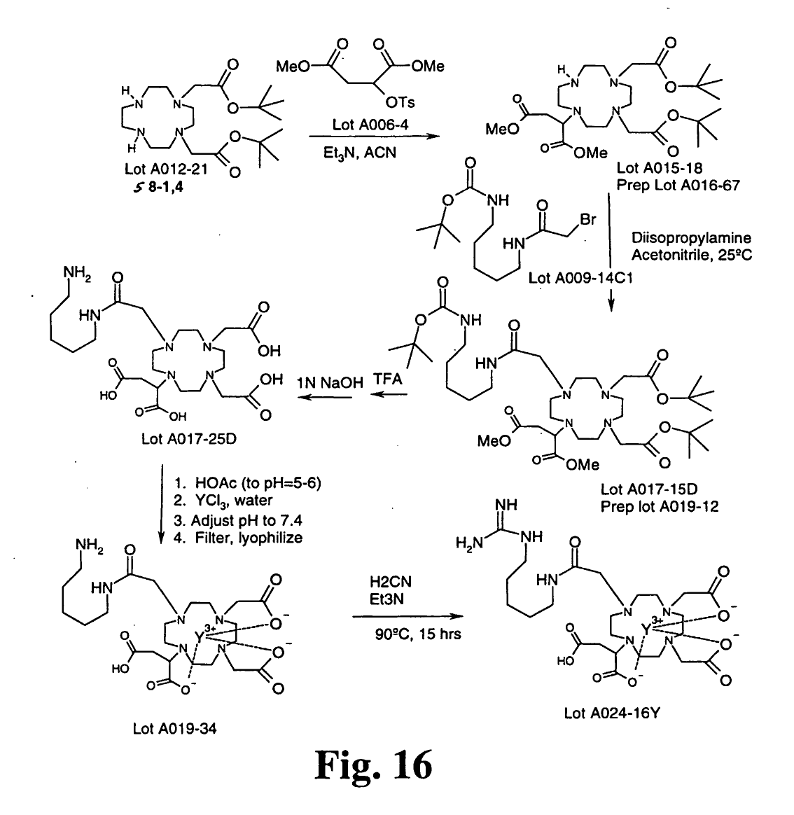

Given that gram quantities of key bis-substituted DO2A as the esters (i.e. structure 58 and 58-1,4 were readily available. a solution phase approach to introducing different substituents was investigated. The inputs made and the processes used to make examples of theses species in gram quantities are schematically shown in FIGS. 14 and 15. The routes started with commercially available materials (represented by 66, 70 and 71). In all about 30 permutations of the amine and carboxy containing side chains and in the 1,4 and in the 1,7 orientation were prepared.

All of these were complexed with yttrium and the tested in the biological assay for their ability to inhibit α_{ν}β₃ mediated adhesion. The results were promising. In particular, one showed significant activity as an RGDS mimetic. The synthesis of this one (Lot A024-16Y) is shown in FIG. 16. The synthetic scheme illustrated in FIG. 16 represents the typical synthesis. Synthesized 58-1,4 was alkylated with the alpha tosylated dimethylsuccinate, which was used because the alpha bromo analog reacted very sluggishly. All of the other alpha bromo alkylations worked fine for this series of compounds. The tri-substituted DOTA was then purified by reverse phase preparative HPLC to give 189 mg of lot A016-67. This lot was then reacted in acetonitrile in the presence of base to give the tetra substituted DOTA analog lot A017-15D which was purified by reverse phase preparative HPLC to give 10 mg (33% yield) of Lot A019-12. The protecting groups were first removed by neat TFA. The TFA was then removed by rotary evaporation and the resulting species was then exposed to aqueous base to saponify the methyl esters (conditions which did not cleave the amide group).

The deprotected tetrasubstituted compound, lot A017-25D was subjected to the complexation conditions to give the yttrium complex characterized by LCMS and assigned lot A019-34. A typical complexation reaction was performed by adding the ligand (which was in basic solution) to hydrochloric acid to bring the pH down to between 5 and 6 and then treating it with an aqueous solution of excess yttrium chloride followed by raising the pH to about 8-9 whereupon the excess yttrium chloride (i.e. that which is not complexed) forms insoluble yttrium hydroxides which can be removed via 0.2 micron filtration. The filtered aqueous solution of the yttrium complexes were then purified in some cases by reverse phase preparative HPLC and lyophilized to give white fluffy solids. The yttrium complexes were characterized by LCMS and gave the proper mass ions.

In order to evaluate whether a primary amine would present a poor bioisostere of guanidine with regard to binding at the α_{ν}β₃ receptor, an analogous primary amine of RGDS (lot A015-82PBS) was prepared. (See the structures below)

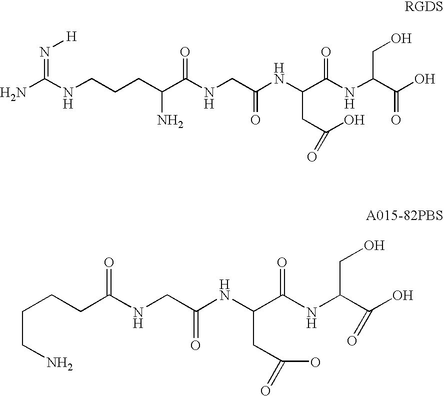

The A015-82PBS species was evaluated in an assay described below and found to be completely inactive even at 100 uM at inhibiting HBEC adhesion. Thus it was determined that the guanidine was an important moiety to include in the preferred species of this invention.

The conversion of the primary amine to a guanidine group was accomplished as illustrated by the conversion of A019-34 to A024-16Y using cyanamide in water at high temperatures (FIG. 16). The final guanidine was characterized by LCMS.

These series of species represented by A024-16Y was tested in the adhesion inhibition assay (see description of the assay below). It was determined that 20 uMolar RGDS decreased the amount of adhesion by 69% (and 100 uMolar decreased it completely) and that compound lot A024-16Y gave a 37% reduction at 100 uMolar. Although exhibiting weaker activity than RGDS, it is the first observation of the ability of a macrocycle with built in acidic and basic functionality to act as a RGD mimic and antagonize the α_{ν}β₃ receptor.

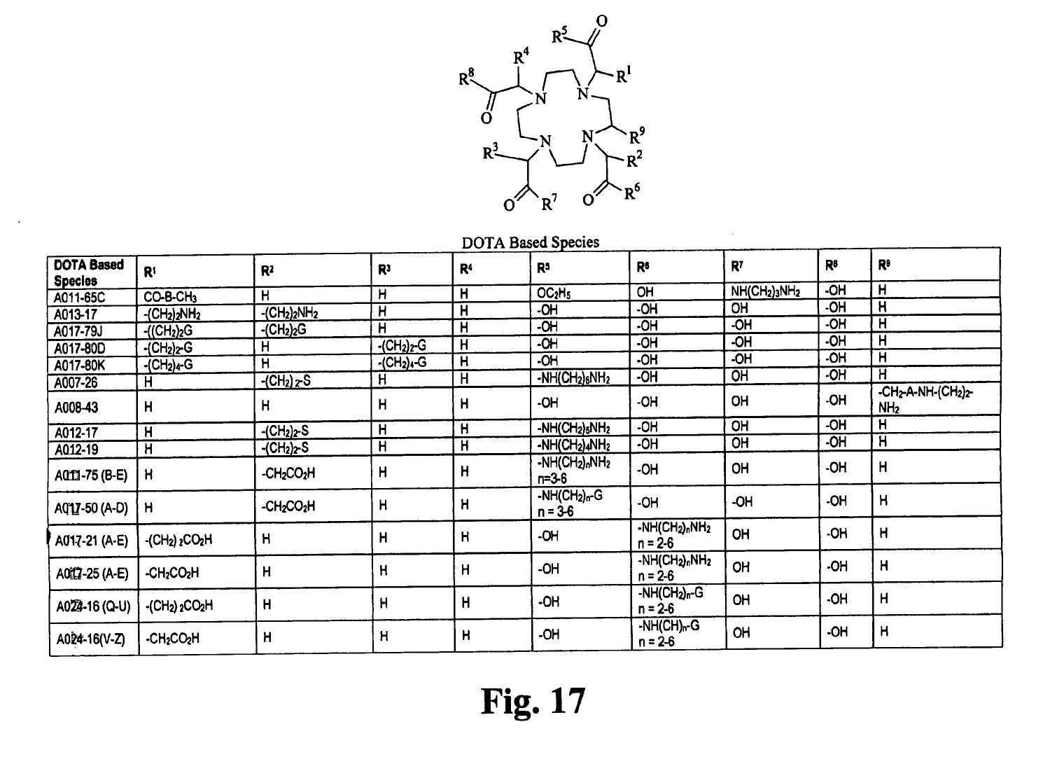

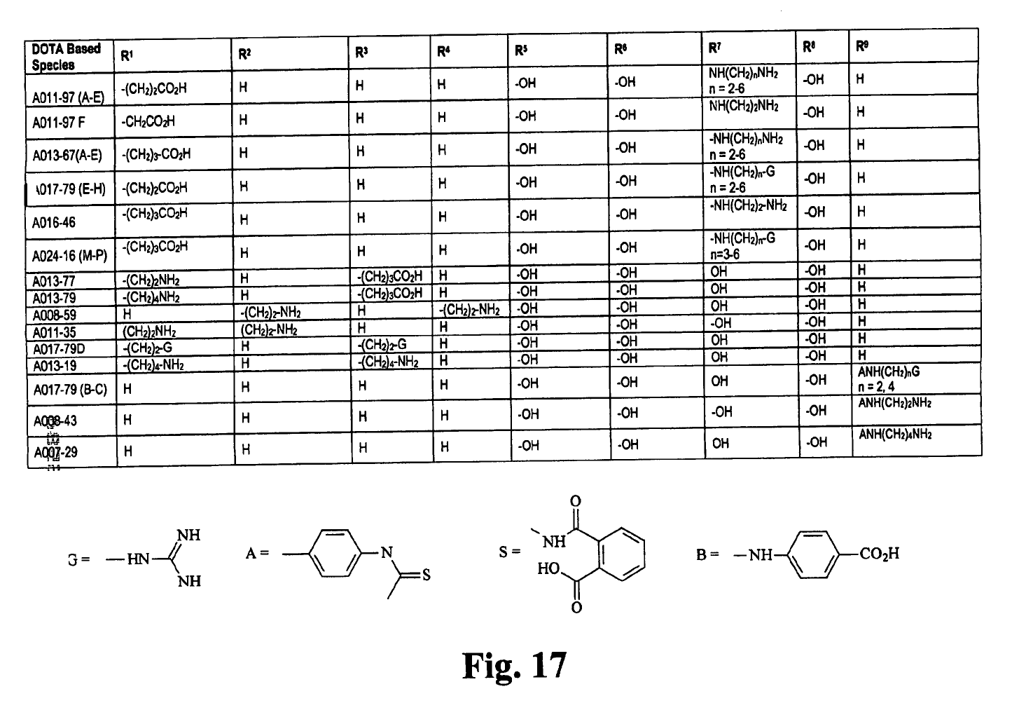

FIG. 17 is a Table listing several examples of DOTA-based scaffolds that can be complexed to metal ions to provide α_{ν}β₃ integrin receptor antagonists in accordance with the present invention.

Biological Assay Studies: A biological whole cell adhesion inhibition assay was developed to evaluate the synthesized macrocyclic RGD mimetics. This assay uses endothelial cells known to express α_{ν}β₃ receptors on their surface (HBEC cells) and also known to require vitronectin binding at the α_{ν}β₃ receptor to initiate adhesion processes. Vitronectin was coated on microtiter plates and exposed to cell suspensions. As the target molecules prepared herein compete with vitronectin for binding at the α_{ν}β₃ they will interfere with the adhesion process. This can be used to quantify/rank their ability to do so. The amount of α_{ν}β₃ mediated adhesion was determined by cell staining with subsequent quantitation by UV absorption proportional to the amount of stain present. This test procedure has been optimized with regard to vitronectin quantities, cell numbers, volumes, times, and the cell staining process. Further the test process was validated with known α_{ν}β₃ antagonists (positive control) and known inactive analogs (negative controls). The IC₅₀ values for known α_{ν}β₃ antagonists were obtained and found to be comparable to those reported in the literature.

### Example of 1,4-DO2A Preparation Using 1:1 Ratio:

It was discovered that if a mixture of alkylated DO2A, DO1A as free bases were allowed to evaporated in a crystallizing dish form methanol/water mixes a white solid would form which is presumable the carbonate salt from the amine reacting with carbon dioxide in water as is known for certain amines. We discovered that the DO1A carbonate salt was very soluble in methanol and thus could be removed from a mixture of DO1A and DO2A as their carbonate salts. We then adjusted the experimental alkylation conditions to take advantage of this separation. A 24.4 mMole portion of cyclen was dissolved in 60 mL of methylene chloride and 20 mL of MeOH. Again 4 eq of triethylamine (13.69 mL) was added all at once. Then via syringe 3.61 mL (24.4 mMoles; 1 equivalent) of t-butyl bromoacetate was added was added all at once. After 12-16 hours the reaction solution was diluted with 40 mL of methylene chloride and extracted with 80 mL of 1N NaOH. The aqueous layer was back extracted with 2×80 mL of methylene chloride and the combined organic layers were dried over sodium sulfate and rotoevaporated to give 5.72 g of clear viscous oil (82% of theory). This 5.72 g was dissolved in 75 mL of MeOH and diluted with 90 mL of water. The solution was poured into a crystallizing dish and allowed to evaporated in a hood until about ⅙th of original volume (about 38 grams total weight) remained (mostly water) and a lot of white solid was present. The solid was filtered and washed with 20 mL water and then dried under vacuum to give 4.3964 g of white solid. By LC-MS analysis this material is 92% bis substituted (in the 1,4 substitution pattern) with 6% mono substituted and only 2% trisubstituted. This form is not suitable for alkylation reactions as it is the carbonic acid salt of the free amine. To convert to the free base a 1.6881 gram portion was dissolved in 12 mL of 1N HCl (about 3 equivalents) and some bubbling as the carbonate is neutralized was noted. Then 2 equivalents (relative to acid added=24 mMoles) of NaOH (6 mL of 4 M NaOH) was added and the free base oils out. The free base was extracted with four 20 mL extractions of methylene chloride. The combined organic layers were dried over sodium sulfate and then decanted and rotoevaporated to yield 1.603 g of pale yellow oil LCMS analysis by spiking this free-base product with an authentic sample of 1,7-substituted material showed two peaks indicating the major product we isolated is indeed the 1,4-bis substituted product (showing a [M+H]+of 401 m/z.).

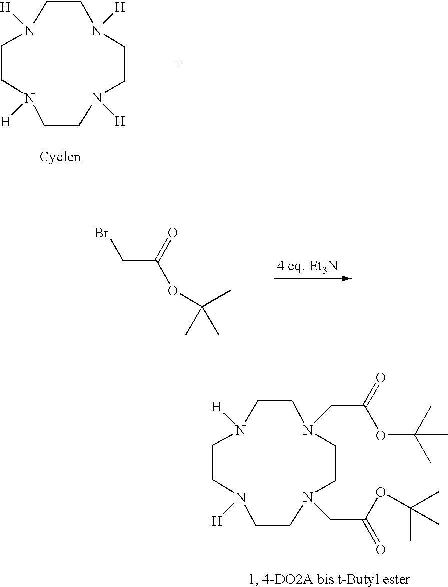

The present invention contemplates modifications as would occur to those skilled in the art. It is also contemplated that complexes, synthetic schemes and treatment methods embodied in the present invention can be altered, rearranged, as would occur to those skilled in the art without departing from the spirit of the present invention. All publications, patents, and patent applications cited in this specification are herein incorporated by reference as if each individual publication, patent, or patent application was specifically and individually indicated to be incorporated by reference and set forth in its entirety herein.

Further, any theory of operation, proof, or finding stated herein is meant to further enhance understanding of the present invention and is not intended to make the scope of the present invention dependent upon such theory, proof, or finding.

While the invention has been illustrated and described in detail in the drawings and foregoing description, the same is considered to be illustrative and not restrictive in character, it is understood that only the preferred embodiments have been shown and described and that all changes and modifications that come within the spirit of the invention are desired to be protected.

### REFERENCES

1. Fan, T. P., Jaggar R., and Bicknell, R. Trends Pharmacol Sci 1995, 16 (2), 57.
2. Folkman, J. Nature Med. 1995, 27.
3. Brown, J. M., and Giaccia, J. A. Cancer Research 1998. 58, 1408.
4. Croix, B. St., Rago, C., Velculescu, V., Traverso, G., Romans, K. E., Mongomery, E., Lal, L., Riggins, G. J., Lengauer, C., Vogelstein, B., Kinzler, K. W. Science 2000, 289, 1197.
5. Kennel, S. J. and Mirzadeh, S. Nuclear Medicine & Biology 1998, 25, 241.
6. Dickman, S. Science 1998, 280, 1196.
7. Barinaga M, Science 1999, 283, 1831.
8. Arap, W., Pasqualini, R., Ruoslahti, E. Science 1998, 279, 377.
9. Pasqualini R, Kolvunen, E., Ruoslahti, E. Nature Biotechnology 1997, 15, 542.
10. Arap, W. Pasqualini, R. Ruoslahti E., Current Opinion in Oncology 1998, 10, 560.
11. Pasqualini, R. Quarterly Journal of Nuclear Medicine 1999, 43, 159.
12. Blok, D. Feitsma, R., Vermeij, P., Pauwels, E. J. K. Eur J. Nucl. Med. 1999, 26, 1511.
13. Volkert, W. A., Hoffman, T. J. Chemical Reviews 1999, 99,2269 and references therein.
14. Seebach, D. and Hoyer, D. Angew. Chem. Int. Ed. 1999, 38, 1223.
15. Hirschmann, R., Nicalaou, K. C., et al. J. Am. Chem. Soc. 1993, 115, 12550.
16. Hirschmann, R., Nicalaou, K. C., et al. J. Am. Chem. Soc. 1992, 114, 9217.
17. Nicolaou, K. C., Trujille, J. I., and Chibale, K. Tetrahedron 1997, 53, 8751.
18. Peczuh, M. W. and Hamilton, A. D. Chem. Rev. 2000, 100, 2479.
19. Haubner, R. et al., J. Nucl. Med. 1999, 40, 1061.
20. Line, B. R., et al. J. Nucl. Med. 1999, 40, 79P, abstract number 319.
21. Keenan, R. M. et al. J. Med. Chem. 1997, 40, 2289.
22. Carron, C. P. et al. Cancer Research 1998, 58, 1930.
23. Haubner, R. et al. J. Nucl. Med. 2000, 41, 42P, abstract number 162.
24. Bock, M. et al. J. Nucl. Med. 2000, 41, 41P, abstract number 160.
25. See references in reference 19.
26. Personal communication, Renata Pasqualini, Nov. 14, 2000.
27. Wong, A. Hwang, S. M., et al. Mol. Pharmacol. 1996, 50, 529.
28. Volkert, W. A., Hoffman, T. J. Chemical Reviews 1999, 99, 2269.
29. Caravan, P. Ellison, J. J. et al. Chemical Reviews 1999, 99, 2293.
30. Molecular modeling was kindly provided by Prof. Lipkowitz and Dr. G. Lombardo both of IUPUI Chemistry Department using the X-ray crystal structure of Y-DOTA complex and applying the universal force field program to arrive at an energy minimized structure (see J. Am. Chem. Soc. 1992, 114, 10024).
31. O'Donnell, M. J., et al. J. Am. Chem. Soc. 1996, 118,6070.
32. Scott, W. L., et al. Tetrahedron Lett. 1997, 38, 3695.
33. Griffith, D. L., et al. Tetrahedron Lett., 1997, 38, 8821.
34. O'Donnell, M. J. et al. Tetrahedron 1999, 55, 6347.
35. Chemical & Engineering News, 2000, October 9, page 52.
36. Mammen, M. et al. Angew. Chem., Int. Ed. 1998, 37, 2754.
37. Burgess, K., and Lim, D., J. Med. Chem. 1996, 39. 4520.
38. Rose, K. J. Am. Chem. Soc. 1999, 121, 7034.
39. Fan, E. et al. J. Am. Chem. Soc. 2000, 122, 2663.
40. Kitov, P. I. et al. Nature, 2000, 403, 669.
41. Smith, J. W., and Cheresh, D. A. J. Biol. Chem. 1988, 263, 18726.
42. Mitjans, F. et al. J. Cell Sci. 1995, 108, 2825.
43. Pytela, R. et al. Science 1986, 231, 1559.

1. A composition comprising:
a metal-chelating ligand including a tetraazacyclododecane macrocycle ring core, and
at least two non-identical substituents covalently bonded to the ring core, wherein each of the at least two non-identical substituents contain a group capable of binding to a cell receptor.

2. The composition of claim 1 wherein at least one of the non-identical substituents is covalently bound to a ring nitrogen.

3. The composition of claim 1 wherein at least one of the non-identical substituents is covalently bound to a ring carbon.

4. The composition of claim 1 wherein at least one of the non-identical substituents are covalently bound to the ring via an alkyl linking group, an alkyl carbonyl linking group, or an alkyl amide linking group.

5. The composition of claim 1 wherein the tetraazacyclododecane macrocycle ring core is chelated to a metal ion.

6. The composition of claim 5 wherein the metal ion is selected from the group of metals consisting of: La, Ce, Pr, Nd, Pm, Sm, Eu, Gd, Tb, Dy, Ho, Er, Tm, Yb, Lu, Y, and Sc.

7. A composition comprising a metal-chelating ligand including tetraazacyclododecane macrocycle having one or more alkyl carboxylic acids or salts thereof appended to the ring nitrogen(s), and
a α_{ν}β₃ receptor binding ligand covalently bonded to a ring nitrogen of the metal-chelating ligand via an alkyl linking group, an alkyl carbonyl linking group, or an alkyl amide linking group.

8. The composition of claim 7 tetraazacyclododecane macrocycle includes two alkyl carboxylic acids or salts thereof each attached to one ring nitrogen.

9. The composition of claim 7 wherein the alkyl carboxylic acid is acetic acid.

10. The composition of claim 7 wherein the alkyl component of the alkyl carboxylic acid or salt thereof is a straight chain, a branched chain, cyclic or aromatic hydrocarbyl group having between 1-5 carbon atoms, and can be substituted with one or more of the following substituents, hydrogen, C1-C4 alkyl, C1-C4 branched alkyl or aromatic or heteroaromatic group or a combination of these groups.

11. The composition of claim 7 wherein the alkyl amide linking group is —(CH₂)ₙCO₂— wherein n is selected to be between 1 and 6.

12. The composition of claim 7 wherein the alkyl component of the alkyl linking group, the alkyl carbonyl linking group and the alkyl amide linking group is —(CH₂)ₙCO₂— wherein n is selected to be between 1 and 6.

13. The composition of claim 7 wherein the alkyl component of alkyl linking group, the alkyl carbonyl linking group and the alkyl amide linking group is a straight chain, a branched chain, cyclic or aromatic hydrocarbyl group having between 1-6 carbon atoms, and can be substituted with one or more of the following substituents, hydrogen, C1-C4 alkyl C1-C4 branched alkyl, aromatic, or heteroaromatic group.

14. The composition of claim 7 comprising a metal ion complexed to the tetraazacyclododecane macrocycle.

15. The composition of claim 7 wherein the metal ion is radioactive.

16. The composition of claim 1 wherein the metal ion is selected from the group consisting of: La, Ce, Pr, Nd, Pm, Sm, Eu, Gd, Tb, Dy, Ho, Er, Tm, Yb, Lu, Y, and Sc.

17. A composition comprising:
a metal-chelating ligand including tetraazacyclododecane macrocycle having one or more alkyl carboxylic acids or salts thereof appended to the ring nitrogen(s), and
a guanidine substituent covalently bonded to a ring nitrogen of the metal-chelating ligand via an alkyl linking group, an alkyl carbonyl linking group, or an alkyl amide linking group.

18. The composition of claim 17 wherein the alkyl component of the alkyl linking group, an alkyl carbonyl linking group or an alkyl amide linking group is a straight chain, a branched chain, cyclic and/or aromatic group.

19. The composition of claim 17 comprising a metal ion complexed to the tetraazacyclododecane macrocycle.

20. The composition of claim 17 wherein the metal ion is radioactive.

21. The composition of claim 17 wherein the metal ion is selected from the group consisting of: La, Ce, Pr, Nd, Pm, Sm, Eu, Gd, Th, Dy, Ho, Er, Tm, Yb, Lu, Y, and Sc.

22. A method of inhibiting tumor cell growth, said method comprising:
administering to the tumor cells an effective amount of a composition including a compound having a metal-chelating ligand including tetraazacyclododecane macrocycle having one or more alkyl carboxylic acids or salts thereof appended to the ring nitrogen(s), and a α_{ν}β₃ receptor binding ligand covalently bonded to a ring nitrogen of the metal-chelating ligand via an alkyl group linking group, an alkyl carbonyl linking group, or an alkyl amide linking group.

23. The method of claim 22 wherein the composition comprises a radioactive metal ion chelated to the metal-chelating ligand.

24. The method of claim 22 wherein the tumor cell is selected from the group consisting of osteosarcomas, neuoroblastomas, glioblastomas, melanomas, and carcinomas.

25. A method of inhibiting tumor cell growth, said method comprising administering to the cells a metal-chelating ligand including tetraazacyclododecane macrocycle having one or more alkyl carboxylic acids or salts thereof appended to the ring nitrogen(s), and
a guanidine substituent covalently bonded to a ring nitrogen of the metal-chelating ligand via an alkyl linking group, an alkyl carbonyl linking group, or an alkyl amide linking group.

26. The method of claim 25 wherein the composition comprises a radioactive metal ion chelated to the metal-chelating ligand.

27. The method of claim 25 wherein the tumor cell is selected from the group consisting of osteosarcomas, neuoroblastomas, glioblastomas, melanomas, and carcinomas.
